(19) United States
(12) Patent Application Publication
(10) Pub. No.: US 2013/0237969 A1
(43) Pub. Date: Sep. 12, 2013

(54) System and Method for Short Scan Interferometric Imaging

(71) Applicants: Kristian Hohla, Muenchen (DE); Holger Schlueter, Muenchen (DE)

(72) Inventors: Kristian Hohla, Muenchen (DE); Holger Schlueter, Muenchen (DE)

(21) Appl. No.: 13/415,661

(22) Filed: Mar. 8, 2012

(51) Int. Cl. A61F 9/008

(52) U.S. Cl. 606/4

(57) ABSTRACT

A system for establishing a reference datum inside the eye of a patient includes an imaging unit for generating and directing imaging beams along respective beam paths into the eye. A detector is connected with the imaging unit and is used to identify the location(s) of marked responses on each beam path where the imaging beam intersects a selected interface surface. A computer then organizes the plurality of marked responses into a predetermined subset according to their common intersection with a same interface surface. This predetermined subset is then fitted with a topology of a surface to establish the reference datum.

### FIELD OF THE INVENTION

The present invention pertains generally to laser systems that are used for ophthalmic surgery. More particularly, the present invention pertains to laser systems that incorporate imaging techniques for use in the guidance and control of a laser beam's focal point relative to a reference datum during ophthalmic surgery. The present invention is particularly, but not exclusively, useful in laser systems that rely on imaging techniques to identify the precise locations of a minimal number of marked responses with which to establish a reference datum for guidance and control of the laser beam.

### BACKGROUND OF THE INVENTION

For an ophthalmic laser surgical procedure, the precise and accurate placement of a laser beam's focal point is of the utmost importance. For example, in most ultrashort pulse, laser ophthalmic procedures, the required placement accuracy of the actual focal point needs to be well within twenty microns of an intended target point. Moreover, this level of accuracy must be maintained throughout the procedure at all times.

Recent advancements of laser technology in the expanding field of ophthalmic applications have brought the operational capability of various laser systems under increasing scrutiny. In particular, along with the precision and accuracy of focal point placement, the time that is required to perform an ophthalmic surgical procedure is of considerable importance. Specifically, the ultimate objective here is to have a procedure that can be performed as quickly as possible.

With specific regard to the time that is involved, it is well known that a so-called “femtosecond” laser system (i.e. a system typically having laser pulses of only a few hundred femtoseconds duration, and typical repetition rates of 10-1000 kHz) is capable of moving its laser beam's focal point with great speed. Speed, however, comes with a price. As indicated above, focal point accuracy must be maintained throughout a procedure, and this requires the focal point to be accurately guided. This, in turn, requires there be an accurately defined reference datum for guiding the laser beam's focal point.

Insofar as ophthalmic laser surgical procedures are concerned, various imaging techniques have demonstrated an efficacy for producing accurate images of anatomical elements inside an eye. In particular, Optical Coherence Tomography (OCT) is an imaging technique that has proven to be particularly efficacious for identifying the interface surface between tissues and/or materials inside an eye that have different indexes of refraction (e.g. anterior chamber fluid and the anterior surface of the crystalline lens). Importantly, it is known that a real time image of such a surface can be very useful as a reference datum for the guidance and control of a laser system during an ophthalmic surgical procedure.

When comparing the capability of a femtosecond laser system for performing an ophthalmic surgical procedure with the capability of an OCT imaging unit for creating a reference datum, the femtosecond laser can be faster. This is particularly so when the reference datum is a three dimensional image of an anatomical interface surface inside an eye, and the reference datum may move. Stated differently, the time required for an OCT imaging system to image an entire interface surface inside an eye is a limiting factor in the operation of a femtosecond laser system. Nevertheless, the present invention recognizes that only selected portions of an interface surface may be needed to establish a reference datum, and that these selected portions can be repetitively imaged with sufficient frequency to complement the capabilities of a femtosecond laser.

With the above in mind, it is an object of the present invention to provide a system and method for precisely identifying a minimal number of OCT responses with which to establish a reference datum for the guidance and control of a laser beam during an ophthalmic laser procedure. Still another object of the present invention is to provide a system and method for repeatedly using selected portions of an anatomical interface surface inside an eye to establish a real time reference datum for the guidance and control of an ophthalmic laser surgical procedure. Yet another object of the present invention is to provide a system and method for establishing a reference datum inside an eye that is easy to implement, and comparatively cost effective.

### SUMMARY OF THE INVENTION

In accordance with the present invention, a system for establishing a reference datum inside the eye of a patient requires the concerted operation of an imaging unit, a detector, and a computer. In detail, the imaging unit is used for generating an imaging beam, and for directing the imaging beam along a beam path into the eye. Preferably, the imaging unit employs Optical

Coherence Tomography (OCT) imaging techniques that will generate a marked response from the imaging beam whenever the beam passes through an interface surface that lies between tissues in the eye having different indexes of refraction. As will be appreciated by the skilled artisan, a marked response will also result when an imaging beam is incident on a non-biological material such as an Intraocular Lens (IOL). In each case, the detector, which is connected with the imaging unit, then identifies the location of the marked response on the beam path. In an operation of the system, several different imaging beams are generated, and a respective number of different marked responses are identified.

Once a marked response has been identified, the computer receives the marked response as input from the detector, and organizes them according to the particular interface surface that caused the response. In this process, marked responses from a same interface surface are organized together and are thereafter used as a subset of marked responses. Specifically, the subset is indicative of the particular interface surface. In addition to the subset of marked responses, the computer also receives a topology of the interface surface as an input. For purposes of the present invention, the topology of a surface will typically be based on diagnostic measurements, patient-related documentation, best assumptions, or the quality requirements of the application. In the event, the computer then fits the known topology of the interface surface to the predetermined subset of marked responses. This fitting thus creates the reference datum. As envisioned for the present invention, subsequent subsets of marked responses are continually generated, at a very fast rate (e.g. 2000 subsets per second), and the particular surface topology is sequentially fitted with each new subset. In this manner, the reference datum can be continuously updated. Further, the reference datum can be established using either a predetermined topology, such as mentioned above, or a so-called “ad hoc” topology that is based solely on marked responses as they are operationally generated in situ. For example, if two or three positions on a corneal surface are measured, only second-order Zernike polynomial coefficients can be accurately calculated. That is, the spherical shape and the cylindrical shape can be determined. If ten points on a surface are measured, then third order Zernike polynomials coefficients can be calculated. If fifteen points on a surface are measured, then fourth order Zernike polynomials coefficients (i.e., defocus, spherical aberration, second order astigmatism, coma and trefoil) can be calculated. Still, depending on the particular level of detail that is required, only a minimum number of required points need to be used.

As envisioned for the present invention, the reference datum is used for the purposes of guiding and controlling a laser beam during an ophthalmic laser surgical procedure. Accordingly, the system will also include a laser unit for generating a laser beam and for focusing the laser beam to a focal point. Further, the system will include a computer program product that will be used by the computer to guide and control movements of the laser beam focal point. Specifically, this guidance and control is accomplished relative to the reference datum during the ophthalmic laser surgical procedure.

In another aspect of the invention, a comparator is connected with the computer for comparing the locations of a first subset of marked responses with the locations of a subsequently identified, second subset of marked responses. Deviations that are detected in the locations of the respective subsets can then be used by the computer to determine whether the reference datum has moved. For instance, movements in translation (x, y, z), tilt or orientation/rotation can be detected. If there has been any movement, an appropriate correction of the laser beam's focal point is made relative to the new location of the reference datum to maintain the integrity of the particular ophthalmic laser surgical procedure. Within this capability, the system of the present invention also effectively functions as an eye tracker.

Although the above disclosure has considered the use of a surface topology for establishing a reference datum, it is to be appreciated that a reference datum can also be arbitrarily established without using a surface topology. For instance, two marked responses can be used to establish a linear reference datum. Three marked responses can be used to establish a circular datum, and so on. It is also noteworthy that in the special circumstance wherein a tissue is constrained to move linearly in one dimension, a single, solitary marked response can be used as the reference datum. In such a case there is obviously no need to fit a surface topology to the single, marked response reference datum. The point here is that, depending on the requirements of a particular ophthalmic laser surgical procedure, a minimal number of marked responses can be used to establish a reference datum that is based on a single point, a collection of points, a line, a collection of lines, a surface, a collection of surfaces or combinations of all the preceding. Importantly, such a reference datum can be established and updated in real time to keep pace with the particular procedure. In all cases, it is an important capability of the present invention that the reference datum can be established with a so-called “short scan OCT” wherein only a minimal number of marked responses are required.

As a practical matter, the system of the present invention can selectively provide information for positioning the focal point of a laser beam in tissue to perform Laser Induced Optical Breakdown (LIOB) of the tissue, for avoiding LIOB in specific areas or volumes of tissue altogether, or for disabling the system as a safety precaution in identifiable situations. As an example of the safety feature provided by the present invention, the shutter or the numerical aperture of the system can be automatically adjusted to insure that LIOB is not inadvertently performed on inappropriate tissue.

In other aspects of the present invention, it will also be appreciated that a same beam scanning device can be used for both the surgical treatment beam and the imaging beam. Further, it is envisioned that the present invention can be used regardless whether the eye is constrained. With this in mind, it will be appreciated that when an eye is unconstrained, the present invention also performs the functionality of an eyetracker and, consequently, a docking apparatus becomes redundant. On still another aspect of the present invention, the location(s) of a marked response(s) can be used for safety purposes to indicate when the surgical laser beam will be outside a planned treatment protocol. In such a circumstance, the present invention can be configured to disable the system.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic layout of the operational components for a system in accordance with the present invention;

FIG. 2 is a three dimensional representation of a plurality of imaging beams and their respective marked responses to the imaging beam from the anatomy of an eye;

FIG. 3 is a three dimensional representation of a subset of the marked responses shown in FIG. 2 when fitted with a topology surface from the eye to establish a reference datum in accordance with the present invention; and

FIG. 4 illustrates the consequent deviations in respective locations of marked responses when the reference datum shown in FIG. 3 is moved.

### DESCRIPTION OF THE PREFERRED EMBODIMENTS

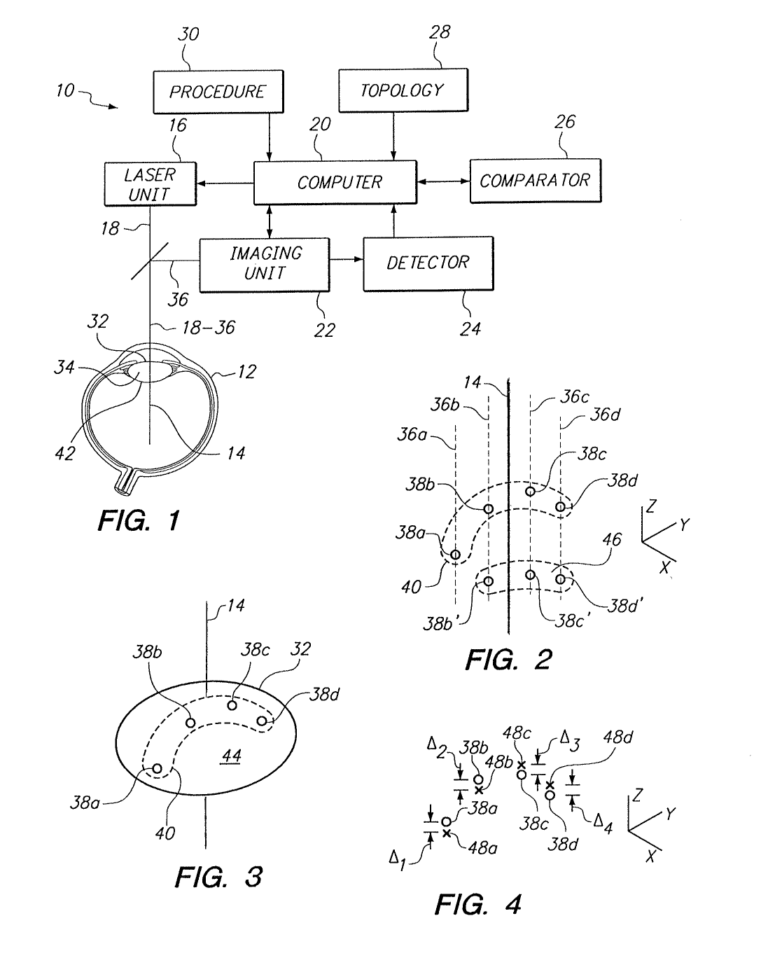

Referring initially to FIG. 1, a system for using imaging techniques in accordance with the present invention to establish a reference datum for use in the guidance and control of a laser beam's focal point during ophthalmic surgery is shown and is generally designated 10. As shown, the system 10 is primarily intended for use in ophthalmic surgery on an eye 12 that defines an axis 14. For purposes of the present invention, the axis 14 can be an optical axis, or a central axis, an arbitrary axis or any other type of defined axis that is well known in the pertinent art. In use, however, the axis 14 is defined for use by a laser unit 16 when directing a laser beam 18 toward the eye 12.

As shown in FIG. 1, the system 10 includes a computer 20 and an imaging unit 22 that is directly connected with the computer 20. For purposes of the present invention, the imaging unit 22 is preferably of a type well known in the pertinent art that is capable of employing the techniques of Optical Coherence Tomography (OCT), topography, Scheimpflug confocal imaging, two-photon imaging, laser (optical) range finding or any other well known imaging modality to include acoustical imaging. Also, the system 10 includes a detector 24 that is connected between the imaging unit 22 and the computer 20. Additionally, a comparator 26 is connected directly to the computer 20. As also shown in FIG. 1, the computer 20 receives inputs that respectively pertain to the topology 28 of a selected anatomical structure inside the eye 12, and to the particular ophthalmic laser surgical procedure 30 that is to be performed. In particular, the topology 28 will pertain to diagnostic measurements (e.g. the topography of the anterior surface 32 of a crystalline lens 34) and/or to patient-related documentation (e.g. the physical characteristics of an implanted Intraocular Lens (IOL) and/or geometric or polynomial shapes selected by a computer or user).

In an operation of the system 10, the imaging unit 22 generates and directs an imaging beam 36 toward the eye 12. Typically, as shown in FIG. 2, the imaging unit 22 will sequentially direct a plurality of imaging beams 36 toward the eye 12, of which the imaging beams 36a, 36b, 36c and 36d are only exemplary. In detail, the imaging beams 36a, 36b, 36c and 36d may be, but not necessarily, parallel to each other, and they will be directed to intersect a known anatomical feature such as the anterior surface 32 of the crystalline lens 34. As is well known, in accordance with OCT imaging, each imaging beam 36 will generate a marked response 38 whenever there is a change in refractive indices between media along its beam path, such as when the imaging beam 36 is incident on the anterior surface 32 of the crystalline lens 34.

By way of example, the imaging beams 36a, 36b, 36c and 36d shown in FIG. 2 are considered to have passed through the anterior surface 32 of the crystalline lens 34, and to have respectively generated marked responses 38a, 38b, 38c and 38d as a consequence. The marked responses 38a, 38b, 38c and 38d are then returned through the imaging unit 22 to the detector 24 where they are identified. From the detector 24 they are sent to the computer 20 where they are collectively evaluated and organized into a subset 40. In this case, the marked responses 38a, 38b, 38c and 38d will all go to the same subset 40 because they all have in common the fact they resulted from the interaction of their respective imaging beams 36a, 36b, 36c and 36d with the anterior surface 32 of the crystalline lens 34. At this point it is noteworthy that the marked responses 38b′ and 38c′ shown in FIG. 2 are indicative of interactions between the imaging beams 36b and 36c and the posterior surface 42 of the crystalline lens 34. These marked responses 38b′ and 38c′, however, are excluded from the subset 40 which pertains to the anterior surface 32 and, instead, will be organized into another subset (not shown). It is to be appreciated that, collectively, the imaging beams 36a-d can be organized as desired for a particular protocol. For instance, the imaging laser beams 36a-d can be organized to collectively lie on a cylindrical surface.

FIG. 3 indicates that the import of the system 10 is to establish a reference datum 44 that can be used by the computer 20 for the guidance and control of the laser unit 16 during an ophthalmic laser procedure 30. As shown in FIG. 3, the reference datum 44 is established using the marked responses 38a-d of subset 40 (see FIG. 2). This is done in accordance with a computer program product of the computer 20. The computer 20 then fits the topology 28 with the subset 40. In this case, the topology 28 pertains specifically to the anterior surface 32 of the crystalline lens 34 which produced the marked responses 38a-d (i.e. subset 40). As will be appreciated by the skilled artisan, the marked responses 38b′, 38c′ and 38d′ shown in FIG. 2 could also be collectively used as a subset 46 that corresponds to the posterior surface 42 of the crystalline lens 34. In this case, another reference datum (not shown) could be established that would be based on the posterior surface 42. Moreover, the subsets 40 and 46 could then each be fitted with respective topologies for the anterior surface 32 and the posterior surface 42 of the crystalline lens 34 and used together for guiding and controlling an ophthalmic laser procedure. It is to be further appreciated that various different reference data can be used for purposes of the present invention (e.g. a cylindrical wall).

It is also envisioned for the present invention that the system 10 can function as an eye tracker. Specifically, as perhaps best appreciated with reference to FIG. 4, the ability of system 10 to quickly generate a subset (e.g. subset 40) allows for a quick comparison of sequential subsets. This comparison will be made by the comparator 26 shown in FIG. 1 and will essentially indicate when a reference datum (e.g. reference datum 44) has moved. For example, consider the marked responses 38a-d of subset 40. With a movement of the eye 12, or with a movement of a tissue within the eye 12, it can happen that each of these marked responses 38a-d will move through a respective deviation Δᵢ, Δ₂, Δ₃ and Δ₄ as evidenced by the marked responses 48a-d. The result here is a new subset (not identified in FIG. 4) that is detected by the system 10 and evaluated with respect to the subset 40 by the comparator 26. With these comparisons, the system 10 is effectively able to compensate for translation in x, y and/or z, as well as tilt and rotation of the eye 12 during an ophthalmic laser surgery.

While the particular System and Method for Short Scan Interferometric Imaging as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A system for establishing a reference datum inside an eye of a patient for use in an ophthalmic laser surgical procedure employing a surgical laser beam focused to a focal point to treat tissue which comprises:
an imaging unit for generating an imaging beam, and for directing the imaging beam along a beam path into the eye;
a detector connected with the imaging unit to identify a location of a marked response from the imaging beam on the beam path; and
a computer for receiving the marked response as input from the detector, and for evaluating this input with a plurality of marked responses from at least one other imaging beam on another beam path to establish the reference datum, and for guiding and controlling movements of the surgical laser beam focal point relative to the reference datum during the ophthalmic laser surgical procedure.

2. A system as recited in claim 1 wherein the reference datum is established using a predetermined subset of marked responses and as many as 2000 subsets are established every second.

3. A system as recited in claim 2 further comprising a comparator connected with the computer for comparing the locations of a first subset of marked responses with the locations of a subsequently identified second subset of marked responses to determine whether the reference datum has moved.

4. A system as recited in claim 2 wherein the reference datum comprises a known topology of a surface, and wherein the surface is fitted to the predetermined subset.

5. A system as recited in claim 4 wherein the known topology of a surface is selected from a group comprising diagnostic measurements, predetermined surfaces and patient-related documentation.

6. A system as recited in claim 2 wherein the reference datum is established solely by marked responses.

7. A system as recited in claim 1 wherein the imaging beam paths are mutually parallel.

8. A system as recited in claim 1 wherein the marked response is indicative of an intersection of a tissue interface with the beam path.

9. A system as recited in claim 1 wherein the marked response is indicative of a location on a non-biological material inside the eye.

10. A system as recited in claim 1 wherein the imaging unit is an Optical Coherence Tomography (OCT) device and wherein the reference datum is established by a complete OCT scan of selected portions inside the eye.

11. A system as recited in claim 1 further comprises:
a laser unit for generating the surgical laser beam and for focusing the surgical laser beam to the focal point; and
a computer program product for use by the computer to guide and control movements of the laser beam focal point relative to the reference datum during the ophthalmic laser surgical procedure.

12. A method for establishing a reference datum inside an eye of a patient for use in an ophthalmic laser surgical procedure employing a surgical laser beam focused to a focal point to treat tissue which comprises the steps of:
generating an imaging beam;
directing the imaging beam along a beam path into the eye;
identifying a location of a marked response from the imaging beam on the beam path;
inputting the marked response to a computer;
using the computer to evaluate the marked response input together with a plurality of marked responses from at least one other imaging beam on another beam path; and
organizing a plurality of the marked responses evaluated in the using step into a predetermined subset of marked responses to establish the reference datum for use by the computer to guide and control movements of the laser beam focal point relative to the reference datum during the ophthalmic laser surgical procedure.

13. A method as recited in claim 12 further comprising the step of comparing the locations of a first subset of marked responses with the locations of a subsequently identified second subset of marked responses to determine whether the reference datum has moved.

14. A method as recited in claim 12 further comprising the step of fitting a known topology of a surface to the predetermined subset to establish the reference datum.

15. A method as recited in claim 14 wherein the known topology of a surface is selected from a group comprising diagnostic measurements, predetermined surfaces and patient-related documentation.

16. A method as recited in claim 12 wherein the directing step is accomplished along a plurality of mutually parallel beam paths.

17. A method as recited in claim 12 further comprises the steps of:
generating a laser beam; and
focusing the laser beam to the focal point.

18. A computer program product for establishing a reference datum inside an eye of a patient for use in an ophthalmic laser surgical procedure employing a surgical laser beam focused to a focal point to treat tissue wherein the computer program product comprises program sections for respectively:
generating an imaging beam; directing the imaging beam along a beam path into the eye; identifying a location of a marked response from the imaging beam on the beam path; inputting the marked response to a computer; using the computer to evaluate the marked response input together with a plurality of marked responses from at least one other imaging beam on another beam path; organizing a plurality of the marked responses into a predetermined subset of marked responses to establish the reference datum; and guiding and controlling movements of the laser beam focal point relative to the reference datum during the ophthalmic procedure.

19. A computer program product as recited in claim 18 further comprising a program section for comparing the locations of a first subset of marked responses with the locations of a subsequently identified second subset of marked responses to determine whether the reference datum has moved.

20. A computer program product as recited in claim 18 further comprising a program section for fitting a known topology of a surface to the predetermined subset to establish the reference datum.

21. A system as recited in claim 1 wherein the reference datum is established using a predetermined subset of at least one marked response.
